(19) United States
(12) Patent Application Publication
(10) Pub. No.: US 2009/0227878 A1
(43) Pub. Date: Sep. 10, 2009

(54) BLOOD PRESSURE METHOD AND DEVICE

(76) Inventor: KIRK K. PETERSON, HUXLEY, IA (US)

Correspondence Address: MCKEE, VOORHEES & SEASE, P.L.C., 801 GRAND AVENUE, SUITE 3200, DES MOINES, IA, 50309-2721, US

(21) Appl. No.: 12/397,512

(22) Filed: Mar. 4, 2009

Related U.S. Application Data
(60) Provisional application No. 61/033,490, filed on Mar. 4, 2008.

(51) Int. Cl. A61B 5/02

(52) U.S. Cl. 600/493

(57) ABSTRACT

An improved device and method is provided for accurately and consistently measuring blood pressure. The device includes a body having an insufflation bulb attached thereto. The bulb is connected to a blood pressure cuff. The cuff can be inflated using the insufflation bulb. A pressure release valve is then opened to slowly relieve pressure from the cuff, while the operator listens for the Korotkoff sounds using a stethoscope. A button on the body of the device is pressed by the operator when the systolic and diastolic sounds are heard. The device automatically records the pressures without the need for a visual reading when the button is depressed.

### CROSS-REFERENCE TO RELATED APPLICATIONS

This application claims priority under 35 U.S.C. §119 of Provisional Application Ser. No. 61/033,490 filed Mar. 4, 2008, which application is hereby incorporated by reference in its entirety.

### BACKGROUND OF THE INVENTION

Blood pressure can be easily taken in a doctor's office or at home using commercially available devices. However, conventional methods for measuring blood pressure often are not accurate due to operator error.

The conventional ausculatory method of measuring or monitoring a patient's blood pressure utilizes a cuff 10 wrapped around the patient's bicep 12, as illustrated in FIG. 1 of the drawings. Preferably, the patient is sitting down, with their arm resting on a table (not shown) so that the brachial artery 13 is level with the heart. If the arm is too high, the blood pressure readings will be low, and if the arm is too low, the blood pressure readings will be too high. The cuff is connected to a gauge, such as a sphygmomanometer 14 or a needle gauge. The cuff is pressurized using an insufflation bulb 16 to a level sufficient to compress the brachial artery so as to preclude blood flow through the artery.

With a stethoscope 18 placed on the hollow of the elbow over the brachial artery, a pressure release valve connected to the bulb or cuff is loosened to slowly release pressure from the cuff 10. Once the systolic pressure is reached (the maximum pressure exerted by the blood against the wall of the brachial artery when the heart beats), the brachial artery opens, allowing volatile blood flow through the artery, which causes vibrations against the artery walls. These vibrations create noise, referred to as the Korotkoff sounds, which are heard through the stethoscope 18. As pressure from the cuff 10 continues to decrease, the blood flow through the brachial artery increases steadily, until the cuff pressure falls below the diastolic pressure (the pressure between successive heart beats), at which time the blood flow through the artery becomes laminar and the Korotkoff sounds cease.

When a sphygmomanometer 14 is used, the liquid in the meter drops steadily, until the systolic pressure is reached, at which time the liquid momentarily stops dropping. Similarly, with a needle gauge, the needle drops steadily until the systolic pressure is reached. The operator must read and remember the markings on the meter or gauge at the point where the liquid or needle momentarily stops. Then, the liquid or needle downwardly drop continues until the diastolic pressure is reached, at which time the liquid or needle momentarily stops. Again, the operator must read and remember the markings on the meter or gauge where the liquid or needle stopped. Then, the operator must record the level for the systolic and diastolic pressures.

These conventional methods and equipment have several disadvantages. First, the markings on the sphygmomanometer 14 and on the needle gauge are small and may be difficult to read. Second, if the patient moves during the process, the fluid in the meter or the needle of the gauge may bounce, thereby resulting in an inaccurate reading by the operator. Also, the operator has to multi-task, including watching the meter or gauge while simultaneously listening with the stethoscope 18 to auscultate for the blood pressure Korotkoff sounds and deflating cuff. Furthermore, the operator has to remember both the systolic and the diastolic measurements before documenting these in a patient record or file. Patient movement may also distract the operator. Furthermore, a gauge needle may bounce prior to the true systolic blood pressure, even without arm movement by the patient, which leads to an inaccurate reading. Inaccurate readings or measurements may result in over medicating problems and unnecessary patient stress.

Home blood pressure kits typically use digital gauges, which also have problems. Digital gauges rely on pressure sensors, which are unable to detect Korotkoff sounds. Such pressure sensors in the cuff are not as accurate as auscultation with a stethoscope. Also, movement of the person's arm can result in inaccurate blood pressure sensing. Home kits also generally come with only one sized cuff, which can produce inaccurate blood pressure readings. These cuffs with pressure sensors are expensive, and not particularly practical for use in a clinical setting. Again, inaccurate measurements may result in over medicating problems and increased patient stress.

The Korotkoff sounds are the sounds heard through the stethoscope as the pressure cuff deflates. The sounds are first heard when the cuff pressure equals the systolic pressure, and cease to be heard once the cuff has deflated past the diastolic pressure.

It is generally accepted that there are five phases of Korotkoff sounds. Each phase is characterized by the volume and quality of sound heard. FIG. 2 of the drawings illustrates these phases. In this example, the systolic and diastolic pressure are 120 mmHg and 80 mmHg respectively.

### Phase 1

When the pressure cuff is inflated to beyond the systolic pressure, the artery is completely occluded and no blood can flow through the artery. Consequently, no sounds are heard with the stethoscope above the systolic pressure. At the point where the cuff pressure equals the systolic pressure, a sharp tapping sound is heard. The blood pressure oscillates between systolic and diastolic pressure. At systolic, the pressure is great enough to force the artery walls open and for blood to spurt through. As the pressure dips to diastolic, however, the artery walls bang shut again. It is the closing shut of the artery walls that results in the tapping sound.

### Phase 2

This phase is characterized by a swishing sound, caused by the swirling currents in the blood as the flow through the artery increases. Sometimes, if the cuff is deflated too slowly, the sounds vanish temporarily. This happens when the blood vessels beneath the cuff become congested, and is often a sign of hypertension. The congestion eventually clears, and sounds resume. The intervening period is called the auscultatory gap.

### Phase 3

In this phase, there is a resumption of crisp tapping sounds, similar to those heard in phase 1. At this stage, the increased flow of blood is pounding against the artery walls.

### Phase 4

At this point, there is an abrupt muffling of sound. The blood flow is becoming less turbulent. Some medical practitioners choose to record this point as the diastolic pressure.

### Phase 5

This is the point at which sounds cease to be heard all together. The blood flow has returned to normal and is now laminar. The pressure cuff is then deflated entirely and removed.

Thus, while blood pressure measurements based upon the Korotkoff sounds is the most accurate method, conventional measurement devices have limitations and problems in providing consistently accurate measurements.

Therefore, a primary objective of the present invention is the provision of an improved blood pressure measuring method and device which overcomes the problems of the prior art.

Another objective of the present invention is the provision of a method and means for accurately and consistently measuring blood pressure.

A further objective of the present invention is the provision of a blood pressure measuring device which is simple to use.

Yet another objective of the present invention is the provision of an improved blood pressure measuring method and means which can be quickly and easily learned for both clinical use and home use.

Still another objective of the present invention is the provision of an improved device for measuring blood pressure using a conventional cuff without pressure sensors.

Another objective of the present invention is the provision of an improved device and method for measuring blood pressure which minimizes chances for error.

Yet another objective of the present invention is the provision of a device and method for measuring blood pressure which allows the operator to focus only on Korotkoff sounds with a stethoscope, and eliminates the need to read the systolic and diastolic pressure readings during the procedure.

Another objective of the present invention is the provision of a device and method for measuring blood pressure which eliminates inaccuracies due to movement of the patient's arm.

A further objective of the present invention is the provision a device for blood pressure measurements having a large digital readout for easy reading.

Another objective of the present invention is the provision of a device for measuring blood pressure which automatically records systolic and diastolic values with the touch of a button.

Another objective of the present invention is the provision of a device for measuring blood pressure which eliminates the need for the operator to remember the systolic and diastolic values during the testing procedure.

Still another objective of the present invention is a device for measuring blood pressure which helps novice operators learn to take more accurate measurements.

Yet another objective of the present invention is the provision of a device for measuring blood pressure which is practical for a patient to use at home and for a trained medical person to use in a clinic, medical office, hospital, nursing home, school, and other settings.

Another objective of the present invention is the provision of an improved blood pressure measuring device which can download the systolic and diastolic pressure values to a computer or other electronic health record.

A further objective of the present invention is the provision of an improved device and method for measuring blood pressure which minimizes over medication problems and minimizes patient stress.

These and other objectives will become apparent from the following description of the invention.

### SUMMARY OF THE INVENTION

The improved blood pressure measurement method and device of the present invention relies upon the Korotkoff sounds for an accurate determination of the systolic and diastolic pressures. The inventive device is hand-held, and utilizes an actuation button which is depressed when the operator hears the initial Korotkoff sound at the beginning of phase I, so as to register the systolic pressure, and depressed again when the Korotkoff sounds stop at the beginning of phase 5, so as to register the diastolic pressure. Operators can be easily trained so as to hit the button immediately upon hearing the first sound and the ending silence, thereby producing a more accurate blood pressure reading than visual observation of a bouncing liquid or needle, or achieved by pressure sensors.

### BRIEF DESCRIPTION OF THE DRAWINGS

FIG. 1 is a schematic showing a prior art blood pressure measurement assembly utilizing a sphygmomanometer.

FIG. 2 is a graph showing the five phases of Korotkoff sounds.

FIGS. 3 and 4 are elevation views from opposite sides of a first embodiment of the blood pressure measurement device of the present invention.

FIG. 5 is a top plan view of the device shown in FIG. 3.

FIG. 6 is a front end view of the device of FIG. 3.

FIG. 7 is a top plan view of a second embodiment the blood pressure measurement device of the present invention.

FIG. 8 is a side elevation view of the device shown in FIG. 7.

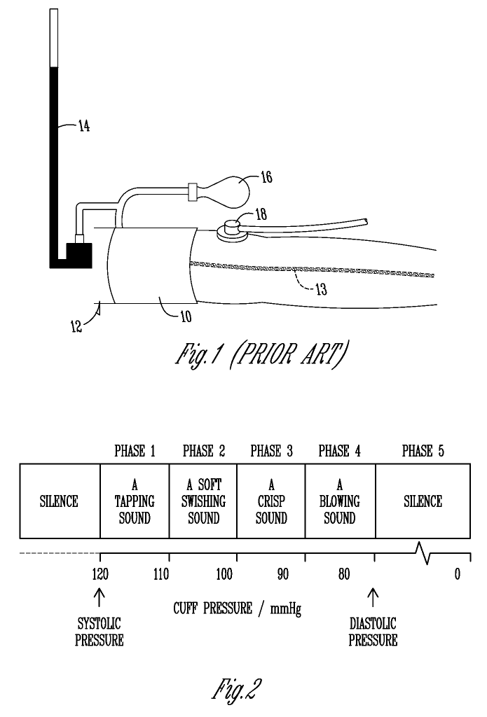

### DESCRIPTION OF THE DRAWINGS

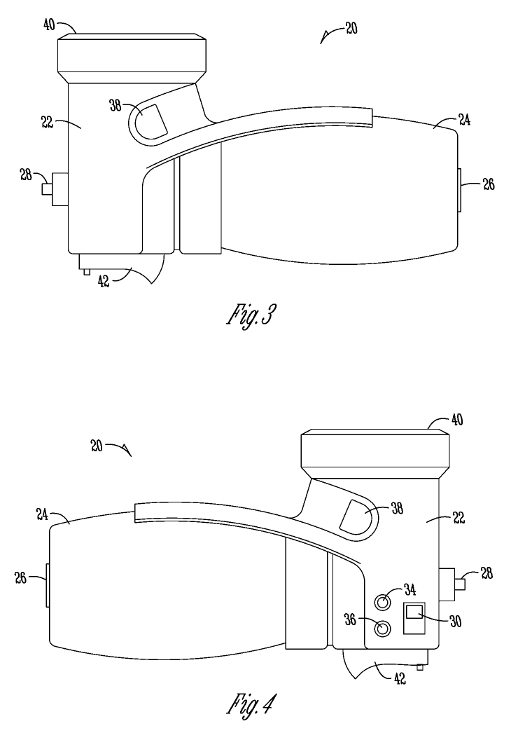

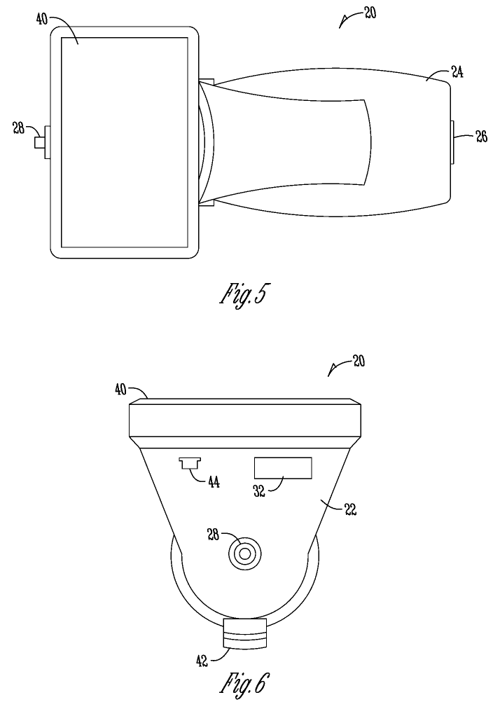

A preferred embodiment of the blood pressure measuring device 20 of the present invention is shown in FIGS. 3-5. The device 20 is easily held in one hand and includes a body 22 with an insufflation bulb 24 mounted on the body 22. The bulb 24 includes an air inlet 26 operatively connectable to a source of pressurized air, and an air outlet or coupling 28 for connecting to the hose of a conventional blood pressure cuff. An on/off button or switch 30 is provided on the body 22. The body 22 also houses one or more batteries (not shown) via an access door 32. The body also includes a pressure input switch 34 which allows an operator or user to set the pressure value at which the insufflation of the cuff will be stopped. The desired pressure setting is selected via another switch or button 36. Preferably, the device 20 includes an audible signal generator which will beep when the pre-selected pressure setting is achieved, for example, at 100 mm HG, 200 mm HG or 300 mm HG.

The device 20 also includes a button 38 which is depressed by the operator to record the systolic and diastolic blood pressures, as described in more detail below. A monitor or display 40 is provided on the device 20 to provide a digital readout of the systolic and diastolic pressures, preferably after the procedure is completed. A pressure relief valve 42 on the device 20 can be easily actuated by the operator to relieve pressure from the cuff to begin the blood pressure reading process. In the embodiment shown in FIGS. 3-5, for a right-handed operator, the button 38 is actuated by the operator's thumb, while the pressure relief valve 42 can be easily triggered using the operator's index finger. The device 20 includes an internal memory to record the systolic and diastolic pressures when the button 38 is depressed by the operator.

The device 20 also includes a port, such as a USB port, to connect the device 20 to a computer or other storage medium to download the systolic and diastolic pressure readings for electronic medical records.

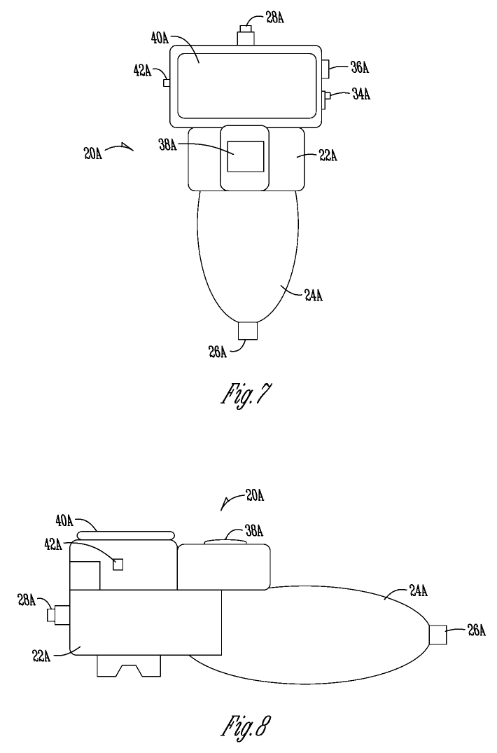

Another embodiment 20A of the device of the present invention is shown in FIGS. 6 and 7. The device of FIGS. 6 and 7 is structurally and functionally similar to the device 20 shown in FIGS. 3-5, with the similar reference numerals being used, with a suffix “A” for the alternative embodiment shown in FIGS. 6 and 7.

The present invention uses a conventional blood pressure cuff 10 of an appropriate size for the patient whose blood pressure is being measured. The insufflation bulb 24 with the digital monitor or gauge 40 of the present invention is connected to the cuff 10 in a convenient manner so that the cuff 10 can be inflated or pressurized by squeezing the bulb 24 in a conventional manner, until the desired cuff pressure which stops the flow of blood through the brachial artery is reached. This initial cuff pressure can be pre-set with the buttons 34, 36 and the device 20 emitting an audible sound when the preset pressure is reached. Alternatively, the device 20 can provide a visual digital readout of the pre-set pressure on the monitor 40 for the operator. Then, the pressure release valve 42 on the device 20 can be opened slightly so as to slowly release pressure from the cuff 10. The readout screen 40 can be turned on so as to show the pressure values as the cuff pressure drops, or may be turned off so that no pressure values are shown. A blank screen is preferable, since it eliminates the need for the operator to visualize the digital readout and allows the operator to focus on the Korotkoff sounds. When the operator hears the initial Korotkoff sound through the stethoscope placed over the brachial artery on the hollow of the patient's elbow, the operator depresses the gauge button 38, which stores the pressure value in a memory of the device 20 for the systolic blood pressure point. When the Korotkoff sounds end, the operator again depresses the gauge button 38 to store the pressure value for the diastolic blood pressure point. The systolic and diastolic pressures can then be displayed on the screen 40 for documentation in a patient file. Alternatively, the device 20 can be coupled to an electronic patient file via the port 44, whereby the systolic and diastolic pressure points are automatically downloaded to the digital file.

The device and method of the present invention provides several advantages over prior art blood pressure measurement techniques and tools. First, the device 20, 20A is simple to use and works with any standard cuff size. The method is simpler by eliminating the need to focus on a sphygmomanometer or needle gauge, and allows the operator to focus only on the Korotkoff sounds so as to produce a more accurate blood pressure reading. Furthermore, movement of the patient's arm during the process does not result in inaccurate measurements, since such movement does not affect the Korotkoff sounds. Also, the digital readout on the monitor 40 is easier to observe than a bouncing liquid in the sphygmomanometer or the bouncing needle gauge. Also, the subjective visual reading by the operator is eliminated by the automatic recording in response to the touch of the button 38. Also, the present invention does not require the operator to remember the pressure point values as the process continues. Thus, the present invention allows new operators to learn and perform accurate blood pressure techniques quicker and easier, both at a clinical setting and at home. The automatic download of blood pressure readings to an electronic file also saves time and expense. The improved accuracy of blood pressure measurements using the present invention also reduces over medication and decreases patient stress, as compared to prior art techniques.

The invention has been shown and described above with the preferred embodiments, and it is understood that many modifications, substitutions, and additions may be made which are within the intended spirit and scope of the invention. From the foregoing, it can be seen that the present invention accomplishes at least all of its stated objectives.

What is claimed is:

1. A method of measuring blood pressure, comprising:
securing a blood pressure cuff around a person's bicep;
pressurizing the cuff to a desired level;
slowly releasing the pressure;
listening for an initial Korotkoff sound using a stethoscope;
pressing a button when the initial Korotkoff sound is heard to register the systolic blood pressure; then
listening for an end of the Korotkoff sound using the stethoscope; and
pressing the button when silence is heard to register the diastolic blood pressure.

2. The method of claim 1 further comprising displaying the systolic and diastolic blood pressures on a monitor.

3. The method of claim 1 further comprising downloading the systolic and diastolic blood pressures into an electronic record.

4. The method of claim 1 further comprising coupling a hand held insufflation bulb to the cuff.

5. The method of claim 4 further comprising mounting a monitor to the bulb and holding the monitor and bulb in one hand of the person taking the blood pressure measurement.

6. The method of claim 5 further comprising providing the button on the monitor whereby the person can actuate the button with their thumb while holding the monitor and bulb.

7. The method of claim 1 further comprising storing the systolic and diastolic pressures in an electronic memory.

8. The method of claim 7 wherein the stored pressures are recalled for recording in a hard copy record.

9. The method of claim 1 wherein the systolic and diastolic pressures are determined without reading a gauge.

10. The method of claim 1 wherein the systolic and diastolic pressures are digitally recorded when the button is pressed the first and second times, respectively.

11. A device for measuring blood pressure, comprising:
a body;
an insufflation bulb on the body and adapted to be attached to a blood pressure cuff via an air line;
a pressure release valve for relieving pressure from the cuff;
a blood pressure recorder in the body to register systolic and diastolic blood pressure; and
an actuation button on the body to actuate the recorder when Korotkoff sounds start and stop.

12. The device of claim 11 further comprising a display monitor on the body to show the systolic and diastolic blood pressure values.

13. The device of claim 11 further comprising a port on the body for receiving a data transmission line to download the systolic and diastolic blood pressures to an electronic file.

14. The device of claim 11 wherein the bulb and body are hand-held.

15. The device of claim 11 further comprising a memory to store the systolic and diastolic pressure values when the button is actuated.

16. The device of claim 15 further comprising a second button on the body to recall the stored pressure values.

17. The device of claim 11 wherein the button is actuated a first time to record the systolic blood pressure and is actuated a second time to record the diastolic blood pressure.

18. The device of claim 11 being free from pressure sensors.
